(19) United States
(12) Patent Application Publication
(10) Pub. No.: US 2015/0096596 A1
(43) Pub. Date: Apr. 9, 2015

(54) SHOWER SANITIZATION SYSTEM AND APPARATUS

(71) Applicants: John James Dvorak, Monroe, CT (US); Mildred Blanche Mendelson, Pound Ridge, NY (US)

(72) Inventors: John James Dvorak, Monroe, CT (US); Mildred Blanche Mendelson, Pound Ridge, NY (US)

(21) Appl. No.: 14/541,048

(22) Filed: Nov. 13, 2014

Related U.S. Application Data
(63) Continuation-in-part of application No. 12/845,093, filed on Jul. 28, 2010, now abandoned.
(60) Provisional application No. 61/277,606, filed on Sep. 28, 2009.

(51) Int. Cl. A61L 2/18; B08B 3/08

(52) U.S. Cl. CPC A61L 2/18; B08B 3/08
USPC 134/7; 134/166 C; 134/93; 134/22.1; 134/8

(57) ABSTRACT

An improved shower or plumbing sanitizing system is provided by a showerhead adapter and/or tap port configured to mount between a water supply stub and an outlet such as a showerhead, hose or faucet and which delivers and imparts a cleansing or sanitizing agent, particularly liquid sanitizer, to the plumbing components prone to infestation with opportunistic bacteria.

### CROSS-REFERENCE TO RELATED APPLICATION

This application is a continuation-in-part of application Ser. No. 12/845,093, filed Jul. 28, 2010, which application claimed the benefit of provisional application Ser. No. 61/277,606, filed Sep. 28, 2009 and which are incorporated herein by reference.

### FIELD OF THE INVENTION

The present invention relates to sanitization improvements for shower systems and related plumbing applications.

### BACKGROUND OF THE INVENTION

The inside of a showerhead is an ideal breeding ground for infectious bacteria. Researchers have identified various microorganisms, including mycobacterium avium, which is known to potentially cause health problems in people who are elderly, pregnant, or otherwise have compromised immune systems. When the shower is turned on, these pathogens not only mix with the bathing water but also become suspended in the air, attached to water droplets. In addition, in use a biofilm is formed on the showerhead parts and accessory parts. When the shower is turned on, various disease-causing microorganisms are released by the water from the biofilm and in addition to mixing with the water, become suspended in the air. Aside from coating articles and surfaces in the bathroom, from towels to toothbrushes, the pathogens can easily be inhaled deeply into the lungs.

The problem arises because the inside of a showerhead or similar plumbing fixture is nearly impossible to thoroughly clean. Most showerheads cannot be readly disassembled and the suggested methods of cleaning, which involve removing the showerhead and boiling it or soaking it in a cleanser are impractical for most people to do on a regular basis.

These and other problems are addressed with the subject sanitizing method and apparatus, which provides a shower sanitizer system that allows the consumer to directly inject cleaner or sanitizer into the showerhead without having to remove it. This method ensures a more thorough distribution of the cleaning solution to the internal parts of the shower hose and head assembly than other methods of cleaning. The end result is a cleaner, safer shower and bathroom. The simplicity and ease of this system will encourage people to clean their showerheads more frequently, and therefore provide greater peace of mind about the health of their family.

In addition to home use, this shower sanitizing system can by used in gymnasiums, hospitals, rehabilitation centers and facilities that cater to people with compromised immune systems. It would also find utility in the hotel industry where present day guests have begun to investigate Internet reviews discussing cleanliness of the properties, and knowledgeable guests are already interested in added value amenities such as special pillows, sheets, and mattresses. Consumers have a heightened awareness of the bacteria and germs in their lives. Those susceptible to infection need to know that something as simple as taking a shower is safe. And those not as susceptible will take satisfaction in knowing that they can easily and thoroughly eliminate bacteria from a part of their home that the family uses every day.

### SUMMARY OF THE INVENTION

The product is a plumbing adapter and/or tap port that fits between the showerhead and water pipe. For ease of installation, in one embodiment, the plumbing adapter can be hand-installed using a simple rubber washer. To start the sanitizing process, the consumer attaches a tube from a bottle of cleaning solution to a one-way flow port on the adapter. This tube attachment can be a screw-on fitting or a ¼ turn engagement for ease of use. After the water is turned on, the cleaning solution is either drawn up the tube and through the adapter as a result of the water pressure, or it may be pumped in via a spray pump on the bottle of solution. A safe and effective cleaning solution will be used, but optionally a marking dye or foaming agent may be added to let consumers know when the entire cleaning agent is out of the showerhead before bathing. After a one-time purchase and installation of the adapter, the cleansing solution would be an ongoing, repurchased product for the system.

A method of cleaning and sanitizing a shower system by providing an easy way for a user to inject a cleansing solution into the water flow upstream of the shower hose or head. This would ensure a more thorough distribution of the cleansing solution to all the internal parts of the shower hose or head assembly. This is to address the constant build up of germs, molds and mildews that develop in a shower system and come out in the water onto the user and into the air. Many people are sensitive to these germs and everyone would want to know that they are showering in clean water. This system would also be valuable to hotel chains, etc for obvious reasons.

An improved sanitization system and a variety of preferred embodiments are provided herein below.

The subject improved sanitization system may be made and used in accordance with the methods detailed below.

Other objects, features and advantages of the present invention will be apparent when the detailed descriptions of the preferred embodiments of the invention are considered with reference to the accompanying drawings, which should be construed in an illustrative and not limiting sense as follows.

### BRIEF DESCRIPTION OF THE DRAWINGS

FIG. 1 is schematic representation of an embodiment of the subject sanitizing system.

FIGS. 2a-d are perspective views of another embodiment of the subject sanitization system.

FIG. 3 is a perspective view of an alternative adapter, which may be used in another embodiment of the subject system.

FIGS. 4a and 4b are perspective views of alternative adapters, which may be used in other embodiments of the subject sanitization system.

FIG. 5a is an end view of a pierce tap which may be used in another embodiment of the subject system.

FIG. 5b is a perspective view of a shower stub combined with the pierce tap shown in FIG. 5a.

### DETAILED DESCRIPTION OF THE PREFERED EMBODIMENTS

The subject improved sanitization apparatus has several important elements as detailed herein below.

There is provided a system comprising an adapter that is configured to mount on a conventional shower stub coming out of a wall. The showerhead or hose would then attach to the adapter. While the sanitization apparatus is described as comprising the adapter, a tap port may be substituted for the adapter with equally good results.

In one embodiment of the subject system, the sanitizing apparatus is comprised of an adapter having standard shower plumbing dimensions and materials and which also provides a tap fitting to which a tube with the appropriate connector would mate to. The tap fitting and connector is generally cylindrical and configured to allow cleansing or sanitizing solution to flow through them when the connector is attached. Further, the port will be sealed shut when the connector is removed; this connection is to be designed for simple hand operation.

In one embodiment, the tube is also connected to a bottle of cleansing or sanitizing solution. When the shower water is turned on, the water stream will draw the solution up and into the water supply. Alternatively, the bottle could incorporate a hand pump, such as on a spray bottle, that would positively inject the solution into the water stream. Any method of delivering the solution to the tap would suffice.

The sanitizing or cleansing solution is to be designed to be effective and safe, should any residual amount remain in the showerhead, and could have an indicator, such as a dye marker or foaming agent added to it to indicate when it is being utilized, and also when it has been rinsed out. The solution is to be an ongoing, repurchased product for use with this system.

Ease of use is facilitated by the subject design. The user would simply fit the tube to a bottle of the cleansing solution, attach the tube connection to the tap fitting, and run the shower while the solution is being injected into the water stream. Cycling the showerhead through its various spray patterns, if it has more than one pattern, would provide thorough cleansing. Running the shower after the tube bas been removed will rinse out the remaining solution.

Alternate methods of providing the delivery system are contemplated including, but not limited to the use of aerosol cans or bottles, single dose systems, pellet systems, powder systems, no-pump aerosol sprays, large spray multi-dose systems, compact pump for travel, or a finger pump. If hotels have adapters pre-installed, travelers can carry their own solution and assure themselves of a clean shower whenever they want. As used herein, the term “liquid” is intended to mean “one of the four primary states of matter, with the others being solid, gas and plasma. A liquid is a fluid. Unlike a solid, the molecules in a liquid have a much greater freedom to move. The forces that bind the molecules together in a solid are only temporary in a liquid, allowing a liquid to flow while a solid remains rigid. A liquid, like a gas, displays the properties of a fluid. A liquid can flow, assume the shape of a container, and, if placed in a sealed container, will distribute applied pressure evenly to every surface in the container. Unlike a gas, a liquid may not always mix readily with another liquid, will not always fill every space in the container, forming its own surface, and will not compress significantly, except under extremely high pressures. These properties make a liquid suitable for applications such as hydraulics.”¹ Alternatively, a liquid is defined as “one of the three fundamental states of matter, along with solids and gases. Unlike a solid (and like a gas) a liquid has constituent particles that are free to move past each other rather than being fixed in a given shape or position. Unlike a gas, it is relatively difficult to compress, and it lacks the capability to expand without limit to fill the space available. Most liquids will assume the shape of a container in which they are confined and will seek the lowest level available. (From a Latin word meaning “to be fluid; flow.”).”² ¹Wikipedia, the free encyclopedia. Liquid. Last modified Sep. 8, 2014, 13:12. <http://en.wikipedia.org/w/index.php?title=liquid>²Definition of “liquid” as set forth in the Dictionary of Science and Technology, Academic Press, Inc., 1992.

Also contemplated are single dose pumps, similar to a caulking gun as well as indicator dyes, soaps or markers of some kind.

Pellet systems can deliver sanitizer to be dissolved each time the shower is used. A lever on the adaptor could route the water through the pellet chamber for cleansing, and when the lever is switched, water would bypass the pellet and go through the showerhead. Gravity or pump injection may also be utilized. To the same effect as the pellet system is a powder system wherein the sanitizer is used as a powder and a lever adaptor is provided to route water through the powder chamber and when the lever is switched, water would bypass the powder and go directly through the showerhead. In the case of pellets, powders and the like, the water routed through the same dissolves in some part the solids or, if small enough the powder and/or pellet particles will be carried along with the water, allowing the liquid containing the dissolved pellets and/or powders or particles thereof to flow through the showerhead.

In other embodiments, bottles of solution stay attached and hang from adapter units near the showerhead and solution may be pumped into water flow with a button or lever attached to the bottle. A user would simply turn on the shower and pump the solution and the showerhead would be clean and rinsed of solution by the time water is warmed up.

A fitting to tap into the shower stub may also be utilized and would be similar to the piercing tap used to supply water to an ice maker in a refrigerator. The fitting has a hardened pierce valve that is driven into the pipe with a hand screw. A washer at the base of the pierce valve seals the pipe once it is seated. A hole through the middle of the pierce valve allows and controls access to the water stream.

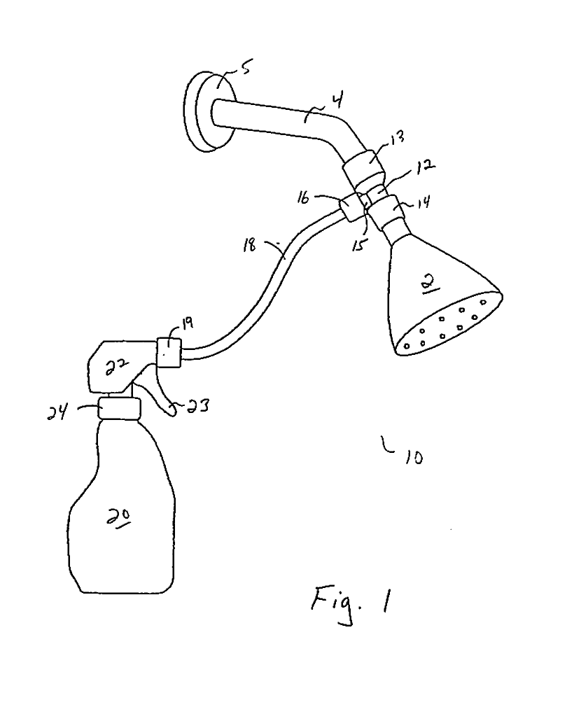

FIG. 1 is schematic representation of an embodiment of the subject sanitizing system 10 depicting a shower stub 4, a sanitizing adapter 12 having stub tap 15, a tube tap connection 16, a showerhead 2 and a bottle for supplying the cleaning agent solution. Sanitizing adapter 12 also has a supply port 15 which connects by tube tap connection 16 with supply tube 18 attached to spray bottle 20 via connector 19. Spray bottle 20 also has a conventional spray head 22 and spray trigger 23 which connects to spray bottle 20 by connector 24. In this figure wall plate 5 is affixed to stub 4, and threaded fitting 14 is used to attach showerhead 2.

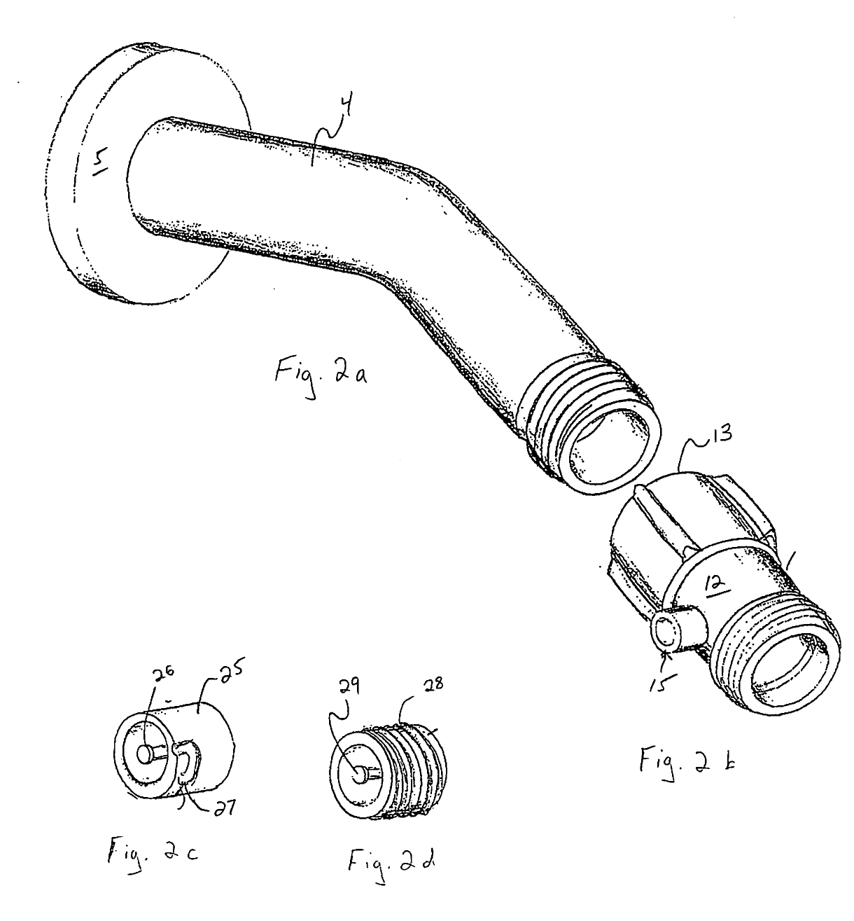

FIGS. 2a-d are perspective views of another embodiment of the subject sanitization system depicting a shower pipe stub 4 which is typically affixed to a shower wall covered by plate 5. In FIG. 2b, a hand tightened slip nut 13 allows proper orientation of adapter 12 and port 15. FIGS. 2c and 2d depict alternate Schrader valves which may be utilized. In FIG. 2c, valve 25 is of the quarter-turn type having detent 27 and actuating mechanism 26. FIG. 2d depicts threaded valve 28 having actuator pin 29.

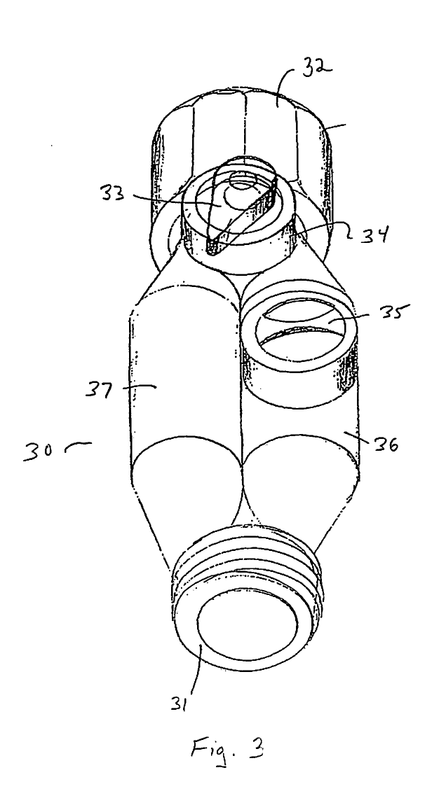

FIG. 3 is a perspective view of an alternative bypass adapter 30, which may be used in another embodiment of the subject system. Depicted is a pellet adapter slip nut 32 to attach to the stub (which allows adapter orientation). Also seen is a chamber hatch 35 with O-ring for inserting a sanitizing pellet, a pellet chamber ball valve selection lever 33 and lever housing 34 to direct flow through the pellet chamber 36 or through a bypass chamber 37, and a threaded fitting 31 for a showerhead.

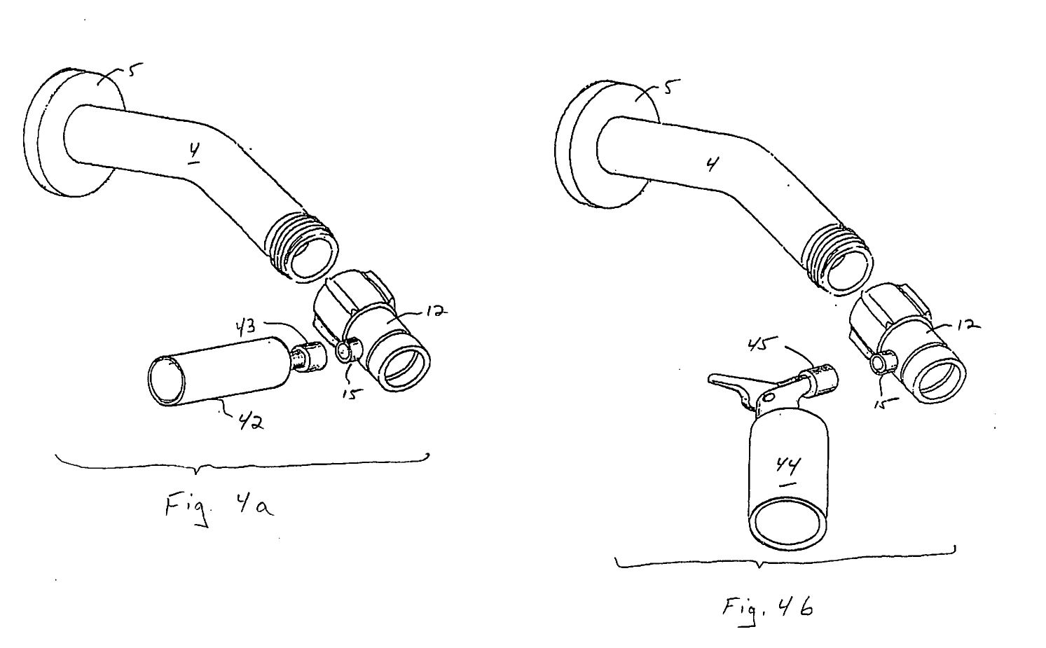

FIGS. 4a and 4b are perspective views of alternative sanitizer supply systems using adapters 12 with stubs 4. In FIG. 4a, supply cartridge 42 is used with supply fitting 43 attached directly to adapter 12 via supply port 15 without need of a supply hose. Similarly in FIG. 4b, pump actuated supply bottle 44 is attached to adapter 12 via supply port 15 with supply attachment fitting 45. FIG. 4a uses a compact aerosol or pump and is actuated by pushing the bottle into the tap.

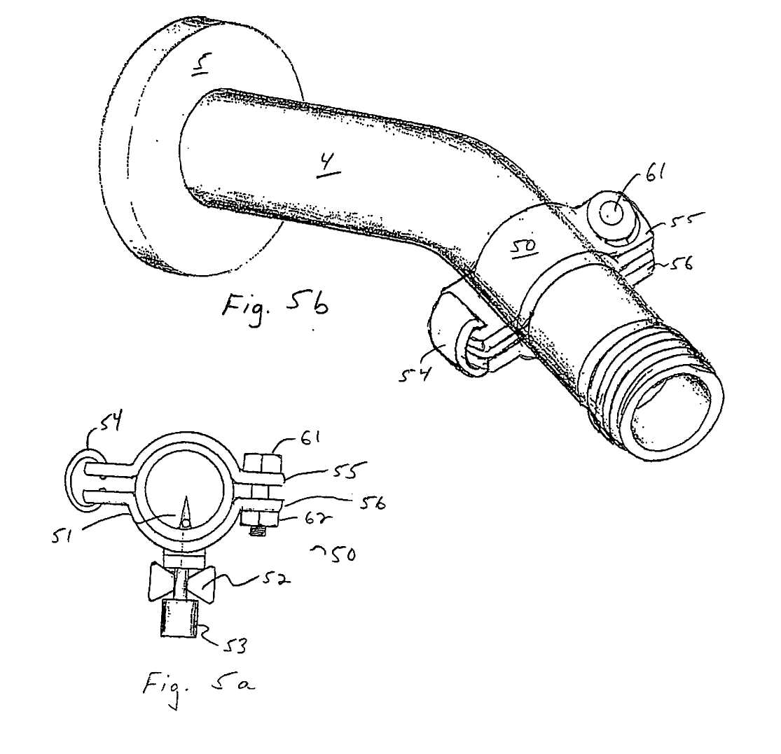

FIG. 5a is an end view of a pierce tap which may be used in another embodiment of the subject system. Depicted is a pierce tap 50 having pierce valve 51 which may be operated in conjunction with a Schrader valve and having adjustable wings 52, supply port 53, adjustment collar 54, tightening bolt and nut 61-62 which maintain opposite valve sides 55 and 56 in position on a pipe stub.

FIG. 5b is a perspective view of a shower stub 4 combined with the pierce tap 50 shown in FIG. 5a.

It will be recognized that the component parts of the subject sanitizing system may be fabricated from metal or plastic parts suitable for use in plumbing applications. Such metals may include aluminum, copper, brass, bronze or steel as may be suitable in individual applications. Suitable thermoplastic resins for use in other applications may include nylons, polyesters, and any of a variety of reinforced thermoplastic resins.

The following examples are provided for illustrating the invention and are not to be construed as limiting the same.

### EXAMPLE 1

Under the direction and control of the applicant, with attention to the details as disclosed and claimed in the above-entitled application, the shower head of the invention was assembled. In addition, after the assembly was complete, the shower head was installed into the bathroom water line for the shower, in order to demonstrate that the nature of the device inserted into the water line shortly before the water is introduced into the shower head was not a matter of choice and that in combination with a shower environment, and soap detergent or other agent having cleansing properties, including water, was effective to substantially, that is almost completely, eliminate the disease carrying bacteria and also the biofilm that contains significant amounts of the aforesaid bacteria. The example was intended to establish that the shower head was filled with a biofilm containing Mycobacterium avium concentrations in amounts relative to other organisms, 100 fold higher than that found in the water before it comes out of the shower head. It is these microorganisms that are known to cause health problems, especially in the elderly, pregnant, or those having compromised immune systems.

Bacteria tend to accumulate forming thin, gooey biofilms inside of the shower heads. When the shower is run, the bacteria and other germs are ejected out of the shower head in the spray. When the fine water droplets are inhaled, Mycobacterium avium gets a direct passage into the lungs, where it proceeds to wreak havoc if the subject's immune system is not strong enough. The example was conducted to ascertain if the device of the invention was under the same conditions effective to reduce the shower head biofilm containing Mycobacterium avium relative to water and/or water containing a cleaning agent. The biofilm from a number of shower heads were sampled and analyzed for ATP and RLU, the former present in all microorganisms, and compared with similar swabs from shower heads but before any water or cleaning agent had entered into the shower head.

The results from the samples which used the device of the invention showed that the biofilm which contains the Mycobacterium avium expressed as ATP contained concentrations of ATP up to 99 fold higher than in the water coming out of the shower head based on swabs taken from the shower head itself.

Some of the particles contained in the water coming from the shower heads were small enough that they could be inhaled into the airways, and which are sprayed onto the body and air around the body. The bacteria and other germs can cause lung infections in both healthy people and those with weakened immune systems. From the results, it can be concluded that shower heads present a significant potential exposure to aerosolized microbes, including documented opportunistic pathogens. The problem which was confronted by the inventor is that the inside of a shower head or similar plumbing fixture is nearly impossible to thoroughly clean and most shower heads cannot be readily disassembled and the suggested methods of cleaning, which involve removing the shower head and boiling or soaking it in a cleanser are impractical for most people to do on a regular basis. The invention provides a shower sanitization system that allows the consumer to directly inject cleaner and sanitizer into the shower head without having to remove it and ensures a more thorough distribution of the cleaning solution to the internal parts of the shower head assembly than other methods of cleaning. The end result, as demonstrated by the decrease in detected microorganisms, is a cleaner, safer shower and bathroom.

In each of the examples reported below, the adapter of the invention was installed between the shower head and the water pipe or line. In the case of the control, in one series of runs, water was used in place of any disinfectant cleaning solution. Samples for analysis were taken by scraping both the interior and exterior of the shower head, both before and after water/water plus detergent were introduced into the shower head and after the water/water plus detergent were allowed to flow through the shower head. The simple technology described above and in the application results in substantial and significant reduction, i.e., elimination, of the microorganisms collected on the outside and inside of the shower head, both before and after the water used in the showering or bathing was introduced into the shower head.

### EXAMPLE 2

As specified in the application, the adapter of the invention is easily installed between the shower head and the water pipe or water line within just a few minutes.

Before initiating the sanitizing process, i.e., installing the adapter of the invention, the face and inside of the shower heads were swabbed with a PocketSwab Plus, manufactured by Charm Sciences, Inc., 659 Andover Street, Lawrence, Mass. 01843, and the RLU reading recorded using the novaLUM, a palm-sized luminometer manufactured by Charm Sciences, Inc., 659 Andover Street, Lawrence, Mass. 01843, capable of reading the PocketSwab Plus test swabs.

The method of the invention was carried out using the device of the invention by:
a. placing the nozzle into the one-way flow port on the adapter;
b. pumping the disinfectant cleaning solution 15 times into the flow port and 6 times on the shower head face, and allowing it to stand for ten minutes; and
c. turning on the shower and allowing the water to run through the showerhead for 30 seconds.

Once the water runs clear and is suds-free, an individual is ready for a safe, clean and healthy shower.

After step c. above (para. [0044]), samples were again taken using a PocketSwab Plus, manufactured by Charm Sciences, Inc., 659 Andover Street, Lawrence, Mass. 01843, which contains an agent capable of cutting through a biofilm and exposing the underlying cells. If a biofilm is present or has developed, it will trigger an elevated RLU count.

Over a two week period, 12 shower heads were tested in New York, Connecticut, and Rhode Island, and included showers installed in national hotel chains. Testing included using three different disinfecting solutions, namely: (1) Oxivir TB, manufactured by Diversey, 8310 16th Street, Sturtevant, Wis. 53177; (2) Oasis 66, manufactured by Ecolab, 370 N. Wabasha Street, St. Paul, Minn. 55102; and (3) Citra Solv, manufactured by Citra Solv, LLC, P.O. Box 2597, Danbury, Conn. 06813. The results of the tests are set forth in Table I below:

TABLE I

| | ATP Before | ATP After | | ATP Before | ATP After | |
|---|---|---|---|---|---|---|
| | (Surface of | (Surface of | % Decrease | (Inside of | (Inside of | % Decrease |
| Location | Shower Head) | Shower Head) | of ATP | Shower Head) | Shower Head) | of ATP |
| National Hotel | 917,145 | 65,185 | 93% | 475,512 | 10,600 | 98% |
| Chain (Rhode | | | | | | |
| Island) | | | | | | |
| Home Shower | 2,812,596 | 121,125 | 96% | 357,265 | 2,449 | 99% |
| (Connecticut) | | | | | | |
| Home Shower | 3,519,498 | 54,859 | 98% | 440,748 | 13,836 | 97% |
| (New York) | | | | | | |

The testing was continued and showed that within 14 days, if left untreated, the biofilm re-growth on showerheads increased by 96%.

As reflected in Table I above, the adapter of the invention allows the disinfectant cleaning solution to get inside the shower head without having to remove it. The invention has the further advantages that it:
a. allows biofilm to be broken down where it forms;
b. prevents consumers from getting a “blast” of bacteria every time they turn on the shower;
c. reduces the spread of infectious bacterial biofilm and microorganisms which are known to cause many health problems, especially for the elderly, pregnant, or those with compromised immune systems; and
d. provides users and businesses offering access to a shower the ability to easily clean their shower heads more frequently, providing freedom from anxiety about the health of their families and customers.

Control values were obtained following the same procedures as set out above. Specifically, two separate .showers were used. Both shower heads were swabbed on the inside and outside. Next, water was run through the shower heads and the shower heads were swabbed again, inside and outside. The adapter was then installed, water and Oxivir solution were used in the cleaning procedure and included a shock step, followed by three (3) maintenance procedures. Water was pumped into the shower head adapter 15 times and 6 times onto the shower head face. The shower head was then allowed to remain at rest for 10 minutes. The shower was then turned on and water allowed to run for 30 seconds. The swabbing procedure was then carried out a third time. The RLU values were then determined, as well as the percentage decrease of ATP. The results of the control testing are set forth in Table II below:

TABLE II

| | | ATP After | | | | | | | | |
|---|---|---|---|---|---|---|---|---|---|---|
| | | Water | | ATP After | | | ATP After | | ATP After | |
| | ATP Before | ONLY(Out- | % De- | Sanitizer | % De- | ATP Before | Water ONLY | % De- | Sanitizer | % De- |
| | (Outside of | side of | crease | (Outside of | crease | (Inside of | (Inside | crease | (Inside of | crease |
| Location | Shower Head) | Shower Head) | of ATP | Shower Head) | of ATP | Shower Head) | Shower Head) | of ATP | Shower Head) | of ATP |
| Control I | 725,344 | 239,531 | 67% | 122,394 | 83% | 555,000 | 345,000 | 38% | 1,533 | 99% |
| (Home | | | | | | | | | | |
| Shower, | | | | | | | | | | |
| NY) | | | | | | | | | | |
| Control II | 965,283 | 540,964 | 44% | 143,270 | 85% | 566,394 | 309,599 | 45.4% | 24,307 | 96% |
| (Home | | | | | | | | | | |
| Shower, | | | | | | | | | | |
| NY) | | | | | | | | | | |

### EXAMPLE 3

In separate and additional tests, the biofilm on the inside and outside of a shower head which had high ATP counts were swabbed. A sample was obtained and sent to BioVir Laboratories, Inc., 685 Stone Road, Unit 6, Benicia, Calif. 94510, specialists in environmental microbiology, in order to determine if the biofilm actually had bacteria growing inside of it. The results which came back were to the effect that the test plates were overgrown with bacteria.

Examples 1 and 2 demonstrate that use of the device before any cleaning procedure and then after cleaning procedures were carried out using Oxivir solution resulted in significant reduction of the ATP value. The reduction in ATP values were significant in and of themselves, establishing the unobviousness of the device of the invention and the manner of its utilization.

The test results before any cleaning procedure was carried out and using water as the cleaning agent are shown in Example 4.

### EXAMPLE 4

The samples listed in Table 4 were submitted to New England Testing Laboratory, Inc., 1254 Douglas Avenue, North Providence, R.I. 02904, on Nov. 21, 2013. The group of samples appearing in the report were assigned an internal identification number (case number) for laboratory information management purposes. The client's designations for the individual samples, along with our case numbers, are used to identify the samples in this report.

TABLE 4-A
Samples Submitted

| Sample ID | Date Sampled | Matrix | Analysis Requested |
|---|---|---|---|
| CT Shower - Home Before | Nov. 20, 2013 | Water | Heterotrophic Plate |
| | | | Count |

The methods as used in carrying out the tests are presented and documented in:
Standard Methods for the Examination of Water and Wastewater, 20^{th} Edition, 1998, APHA, AWWA-WPCF.
Manual of Methods for Chemical Analysis of Water and Water Wastes, EPA-600/4-29-020 (Revised 1983), USEPA/EMSL.
40 CFR 136, Guidelines Establishing Test Procedures for the Analysis of Pollutants Under the Clean Water Act, Office of Federal Register National Archives and Records Administration.
EPA-821-B-94-004

This report of analytical results pertains only to the sample(s) provided to declarant herein and/or by someone who was working under his control and/or supervision, and are so indicated on the custody record.

All samples were found to be properly preserved/cooled upon receipt. All analyses were performed within. EPA designated holding-times. The procedure/calibration checks required by the designated protocols were within the control limits.

TABLE 4-B
Results

| Parameter | Result, CFU/ml | Reporting Limit | Date Analyzed |
|---|---|---|---|
| Heterotrophie Plate | >57,000 | 1 | Nov. 21, 2013 |
| Count | | | @ 15:15 |

The samples listed in Table IV were submitted to New England Testing Laboratory on Nov. 26, 2013. The group of samples appearing in the report was assigned an internal identification number (case number) for laboratory information management purposes. Our designations for the individual samples, along with our case numbers, were used to identify the samples in this report.

TABLE 5-A
Samples Submitted

| Sample ID | Date Sampled | Matrix | Analysis Requested |
|---|---|---|---|
| CT Shower - Home After | Nov. 25, 2013 | Water | Heterotrophic Plate |
| | | | Count |

The source of the methods used are the same as in paragraph 54, above.

All of the samples were found to be properly preserved/cooled upon receipt. All analyses were performed within EPA designated holding-times. Procedure/calibration checks required by the designated protocols were within control limits.

TABLE 5-B
Results

| Parameter | Result, CFU/ml | Reporting Limit | Date Analyzed |
|---|---|---|---|
| Heterotrophic Plate | 18 | 1 | Nov. 26, 2013 |
| Count | | | @ 17:45 |

From the results expressed in Tables III-B and IV-B as set forth above, a decrease from >57,000 CFU/ml (colony-forming units per milliliter) to 18 CFU/ml, it can be appreciated that the device of the invention significantly reduces the biofilm buildup, therefore reducing the bacteria growing inside of the shower head.

Use of the device in the experiments before any cleaning procedure and then after cleaning procedures were carried out using Oxivir solution, resulted in significant reduction of the ATP value. The reduction in ATP values was significant in and of itself, establishing the unobviousness of the device of the invention and the manner of its utilization.

There is evidence which suggests that specific members of HPC bacteria found in drinking water may be causative agents of both hospital- and community-acquired infections. However, the case numbers may be very low and risks represent levels generally less than 1/10,000 for a single exposure to the bacterial agent. The number of cases of pulmonary diseases associated with Mycobacterium avium is rapidly increasing (Rusin et al. 1997) and in some areas is approaching the incidence of Mycobacterium tuberculosis. The epidemiological significance of this waterborne opportunistic pathogen is still not clearly defined (Anaissie et al. 2001).³ ³ M. Exner et al. Public health aspects of the role of HPC—an introduction. World Health Organization, 2003.

Furthermore, the operation and cleaning of devices, particularly medical devices using drinking-water, need to be taken into account. Contamination of these devices with waterborne heterotrophic microorganisms can lead to multiplication of microorganisms (regrowth) in the devices, leading to a significant risk of infection (e.g., by inhalation or endoscopes). The cooling water of dental units, which is sprayed into the patient's mouth, is also often heavily contaminated with Pseudomonas aeruginosa.⁴ <www.who.int/water_sanitation_health/dwq/HPCFull.pdf>. (accessed Feb. 22, 2014). ⁴ Id.

Under such conditions, even low concentrations of certain heterotrophic microorgansims, particularly for people on antibiotics, with immunosuppression or with invasive devices, can be sufficient to cause serious infectious complications.

The invention now being fully described, it will be apparent to one of ordinary skill in the art that many changes and modifications can be made thereto without departing from the spirit or scope of the invention set forth herein.

1. An apparatus for sanitizing plumbing fixtures by elimination of microorganisms in a plumbing fixture, said plumbing fixture adapted for allowing the delivery of cleaner or sanitizer directly into said plumbing fixture without having to pass through the water supply line, comprising: a plumbing fixture, a sanitizing adapter or tap port, a water supply line, said sanitizing adapter or tap port being configured for attachment at one end to said water supply line and adapted for attachment at a generally opposite end of said sanitizing adapter or tap port to a plumbing fixture, said sanitizing adapter or tap port having a supply port for delivering a sanitizing or cleansing agent to a central body of said sanitizing adapter, water from said water supply flowing through said adapter and a mechanical control mechanism which can be activated for preventing the supply of said sanitizing or cleansing agent to the water supply line so that the microogranisms in said plumbing fixture do not mix with the water supplied from said water supply line to said plumbing fixture or become suspended in the air.

2. An apparatus as in claim 1 wherein said sanitizing adapter or tap port is attached to said water supply line by a threaded attachment.

3. An apparatus as in claim 1 wherein the plumbing fixture is a showerhead, shower hose or faucet.

4. An apparatus as in claim 1 wherein the sanitizing or cleansing agent is a liquid sanitizing or cleansing agent.

5. An apparatus as in claim 4 wherein the sanitizing or cleansing agent additionally contains at least one member selected from the group consisting of deodorizing agents, antimicrobials, anti-infectives, anti-molds and surfactants.

6. An apparatus as in claim 1 wherein the sanitizing adapter is a bypass adapter.

7. An apparatus as in claim 1 wherein said tap port is attached at one end thereof to said water supply line and at the other end thereof to a plumbing fixture.

8. An apparatus as in claim 5 wherein the sanitizing or cleansing agent is a solid pellet or powder sanitizing or cleansing agent in a liquid.

9. An apparatus as in claim 1 wherein the supply port for delivering a sanitizing or cleansing agent to the central body of said sanitizing adapter or tap port has a supply tube connected to a bottle for containing a liquid sanitizing or cleansing agent.

10. An apparatus as in claim 1 wherein the supply port for delivering a sanitizing or cleansing agent to the central body of said adapter has a mechanical control mechanism for preventing the supply of sanitizing agent to the water supply line.

11. An apparatus as in claim 9 wherein the mechanical control mechanism is a Schrader valve.

12. A method of sanitizing a plumbing fixture comprising the steps of attaching a sanitizing adapter and/or tap port between a water supply line and a generally opposite plumbing fixture, wherein the adapter and/or tap port has a supply port for delivering a sanitizing or cleansing agent to the central body of the sanitizing adapter or tap port and a water supply flowing therethrough and a mechanical control mechanism for permitting or preventing the supply of sanitizing agent to the water supply line; adding a sanitizing or cleansing agent to the water supply; and thereby sanitizing or cleansing the plumbing fixture.

13. A method as in claim 12 wherein the sanitizing or cleansing agent is a liquid sanitizing or cleansing agent.

14. A method as claimed in claim 13 wherein the sanitizing or cleansing agent additionally contains at least one member of the group consisting of deodorizing agents, antimicrobials, anti-infectives, anti-molds and surfactants.

15. A method as in claim 12 wherein the sanitizing adapter is a bypass adapter.

16. A method as in claim 12 wherein a tap port is attached between the water supply line and the plumbing fixture.

17. A method as in claim 14 wherein the sanitizing or cleansing agent is a solid pellet or powder-form sanitizing or cleansing agent.

18. A method as in claim 12 wherein the supply port for delivering a sanitizing or cleansing agent to the central body of the adapter has a supply tube connected to a liquid sanitizing or cleansing agent.

19. A method as in claim 12 wherein the supply port for delivering a sanitizing or cleansing agent to the central body of the adapter has a mechanical control mechanism for permitting or preventing the supply of sanitizing agent to the water supply line.

20. A method as in claim 18 wherein the mechanical control mechanism is a Schrader valve.

21. An apparatus as in claim 4 wherein the sanitizing or cleansing agent is introduced as a solid pellet or powder and is supplied to said supply port of said sanitizing adapter or tap port from a bottle or container adapted to formulate a sanitizing or cleansing agent in situ by mixing said pellet or powder with water and delivering the liquid sanitizing or cleansing agent formed to said sanitizing adapter or tap port.
